(19) United States
(12) Patent Application Publication
(10) Pub. No.: US 2013/0345768 A1
(43) Pub. Date: Dec. 26, 2013

(54) AUTOMATED EXTERNAL DEFIBRILLATOR WITH LIMITED CPR PAUSE

(71) Applicant: Gintaras Vaisnys, Chicago, IL (US)

(72) Inventor: Gintaras Vaisnys, Chicago, IL (US)

(21) Appl. No.: 13/531,671

(22) Filed: Jun. 25, 2012

(51) Int. Cl. A61N 1/39

(52) U.S. Cl. 607/5

(57) ABSTRACT

A method for administering a therapeutic shock wherein the therapeutic shock is delivered within a pre-determined pre-shock pause period. In an exemplary embodiment, the method is implement on an AED.

### TECHNICAL FIELD

The present invention relates to automated external defibrillators (AED), and, more specifically, to a method of AED operation wherein a pre-shock pause period, the time from ceasing cardiopulmonary resuscitation (CPR) to delivery of a therapeutic shock, is limited.

### BACKGROUND OF THE INVENTION

External defibrillators are emergency medical devices designed to supply a controlled electric shock (i.e., therapy) to a person's (e.g., victim's) heart suffering cardiac arrest. This electric shock is delivered to the heart via pads that are electrically connected to the external defibrillator and in contact with the person's body.

To provide a timelier rescue attempt for a person experiencing cardiac arrest, some external defibrillators have been made portable, by utilizing battery power (or other self-contained power supplies), and many have been designed to be operated by non-medical personnel, who may not even have minimal training. These external defibrillators are more commonly referred to as automated external defibrillators (AEDs), which are available in two types—automatic and semi-automatic.

As many AEDs are designed for operation by non-medical personnel, AEDs autonomously perform a medical analysis on a victim to determine if a defibrillation shock should be delivered. To perform this medical analysis, the AED ascertains the victim's heart rhythm and then analyzes it, using a computer program running on the AED. If the analysis determines the heart rhythm is a “shockable” rhythm¹, a therapeutic shock will be authorized. For an automatic AED, the therapeutic shock will be automatically delivered, while for a semi-automatic AED, the AED will request a therapeutic shock be given, which is typically accomplished by a rescuer pushing a button located on the AED. ¹A “shockable” rhythm is a heart rhythm that can potentially be converted from is present rhythm into a normal rhythm. Not all life threatening non-normal heart rhythms are considered “shockable” rhythms.

In a typical rescue situation where an AED would be appropriate, CPR is an integral part of the rescue attempt. Ideally, CPR will be begun while someone else obtains the AED.

Unfortunately, CPR conflicts with the AED's ability to obtain analyzable heart rhythm data; thus, complicating the AEDs ability to make a determination as to whether the victim's heart has a “shockable” rhythm. While various methods have been proposed to simultaneously administer CPR and obtain analyzable heart rhythm data, generally AEDs require CPR to cease for a period of time.

Recent studies have indicated that an extended pre-shock pause period, the time from when CPR is stopped until a therapeutic shock is administered, can decrease the chances of a successful outcome (re-establishment of a normal heart rhythm). These studies indicate that the pre-shock pause period should not exceed 20 seconds and ideally should be under 10 seconds.

It should be appreciated, that for an AED's rescue programming to obtain heart rhythm data and analyze the data, can take up to 10 seconds. In addition, some AEDs, to maximize battery life, only fully charge after an affirmative therapeutic shock decision has been made (i.e., the victim has been determined to have a “shockable” rhythm). Where this is the case, charging with a strong battery can take 4 to 9 seconds, but where the AED has weak batteries, charging can take significantly longer. As those skilled in the art will appreciate, even where an AED does not employ partial charging prior to an affirmative therapeutic shock decision, an AED with weak batteries will require longer to charge than one with strong batteries.

In addition, for a semi-automatic AED where a therapeutic shock is merely requested, the delivery of the therapeutic shock could be delayed due to hesitation of the rescuer.

What is needed in the art is a method of AED operation that accounts for the critical pre-shock pause period. More precisely, the AED rescue programming must be capable of instructing an operator in such a manner that a therapeutic shock is delivered in a timely fashion relative to when CPR had been terminated.

Furthermore, other desirable features and characteristics of the present invention will become apparent from the subsequent detailed description of the invention and the appended claims, taken in conjunction with the accompanying drawings and this background of the invention.

### SUMMARY OF THE INVENTION

The invention is an AED incorporating additional programming in its conventional rescue programming that attempts to assure that a therapeutic shock is delivered within a pre-determined pre-shock pause period.

Other features, attainments, and advantages will become apparent to those skilled in the art upon a reading of the following descriptions when taken in conjunction with the accompanying drawings.

### BRIEF DESCRIPTION OF THE DRAWINGS

FIG. 1 is a typical rescue timeline.

FIG. 2 is a top view of an illustrative AED on which the present invention may be used.

FIG. 3 is a perspective side view of the AED depicted in FIG. 2

FIG. 4 is a functional block diagram of the components of the AED depicted in FIGS. 1 and 2.

FIG. 5 is a flow chart of a first embodiment of a pre-shock pause program.

FIG. 6 is a flow chart of a second embodiment of a pre-shock program.

### DETAILED DESCRIPTION OF EXEMPLARY EMBODIMENTS

As used herein the term “pre-shock pause period” means a period in which a therapeutic shock must be delivered, wherein the period begins when CPR is stopped, in fact or assumed.

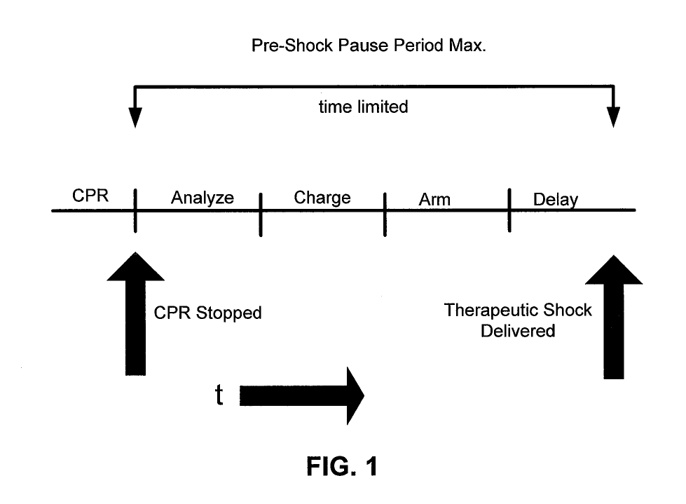

FIG. 1 depicts a rescue sequence with a pre-shock pause period shown. In this illustrative rescue sequence, the pre-shock pause period assumes that for the AED unit to analyze a victim's heart rhythm to determine if a shockable rhythm is present, CPR must cease. Additionally, the pre-shock pause period may include time to charge the AED unit to deliver the therapeutic shock, time to “arm” the unit to deliver the therapeutic shock, and time to deliver the therapeutic shock. As those skilled in the art will appreciate, the activities conducted during the pre-shock pause period may vary as well as the allocations of time to these illustrative activities depending upon the capability of the AED unit and its associated rescue programming.

Depending upon the capabilities of an AED's rescue programming, stopping CPR could be determined in fact or assumed. More specifically, where the AED's rescue programming is capable of determining whether CPR is being performed, the stopping of CPR could be determined in fact. Where the AED's rescue programming is incapable of determining whether CPR is being performed, the proper administration of CPR is assumed; thus, the stopping of CPR upon command to do so is assumed. For example, it is assumed that CPR is commenced when the AED rescue programming directs a rescuer to perform it, and it is discontinued then the AED rescue programming directs the rescuer to stop.

Optionally, the pre-shock period may begin immediately upon turning the AED ON. More specifically, present rescue protocol dictates an AED deliver a therapeutic shock as quickly as possible after the AED is turned ON. Thus, the AED at this point in the rescue sequence never directs the user to perform CPR. As a result, when the AED is turned ON, it may be assumed that CPR, if being performed at all, has just stopped. As a result, the pre-shock pause period in this situation would be the maximum amount of time permitted between turning the AED ON and delivering a therapeutic shock.

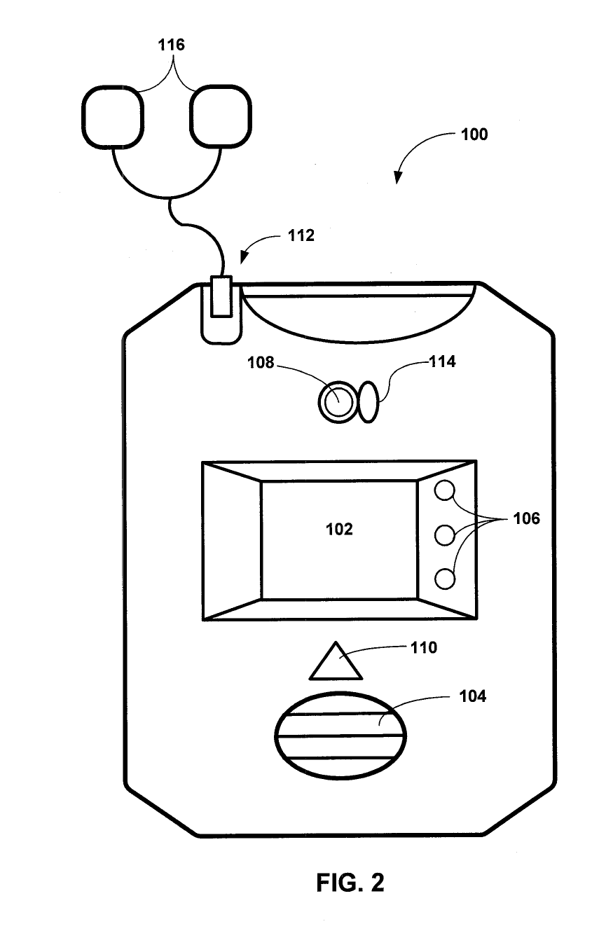

Turning now to FIG. 2, FIG. 2 illustrates a plan view of a semi-automatic AED 100 having a video display 102, a speaker 104, an audio output jack 105, and a user interface 106. The AED 100 further includes an ON/OFF switch 108, a shock switch 110, a pad connector 112, and an active status indicator 114 (e.g., a light source which blinks green indicating in STANDBY sub-mode (discussed below) and operating normally, solid green indicating in ON mode (discussed below) and operating normally, solid red indicating in ON mode with a problem, and blinking red indicating in STANDBY sub-mode with a problem). The pad connector 112 connects pads 116 to the AED 100.

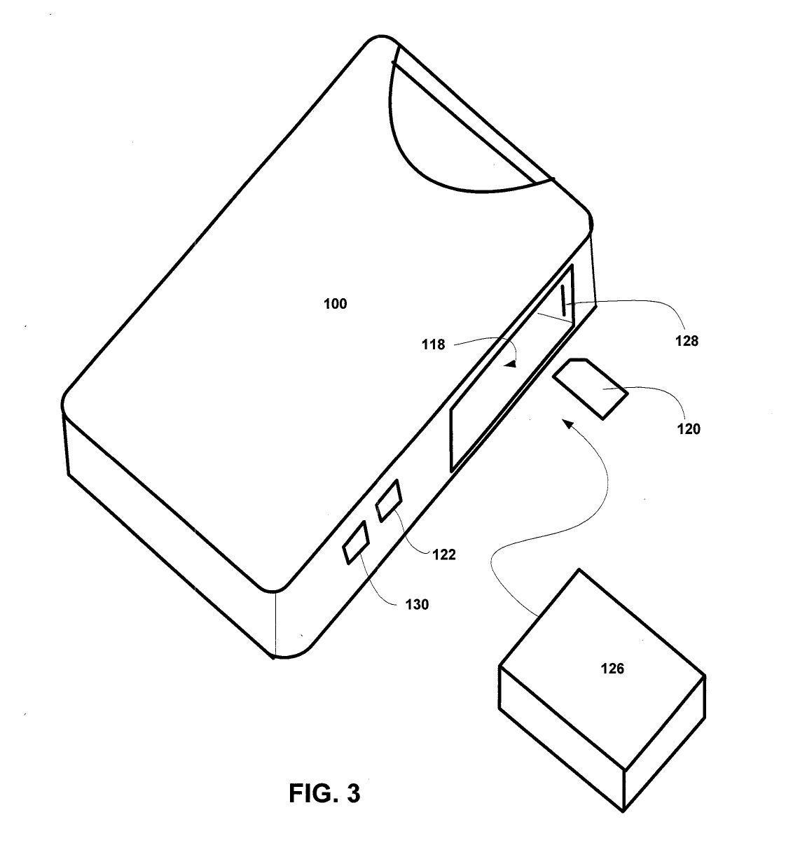

Referring to FIG. 3, the AED 100 includes a battery 126 (removed from AED for clarity) that provides the main power.

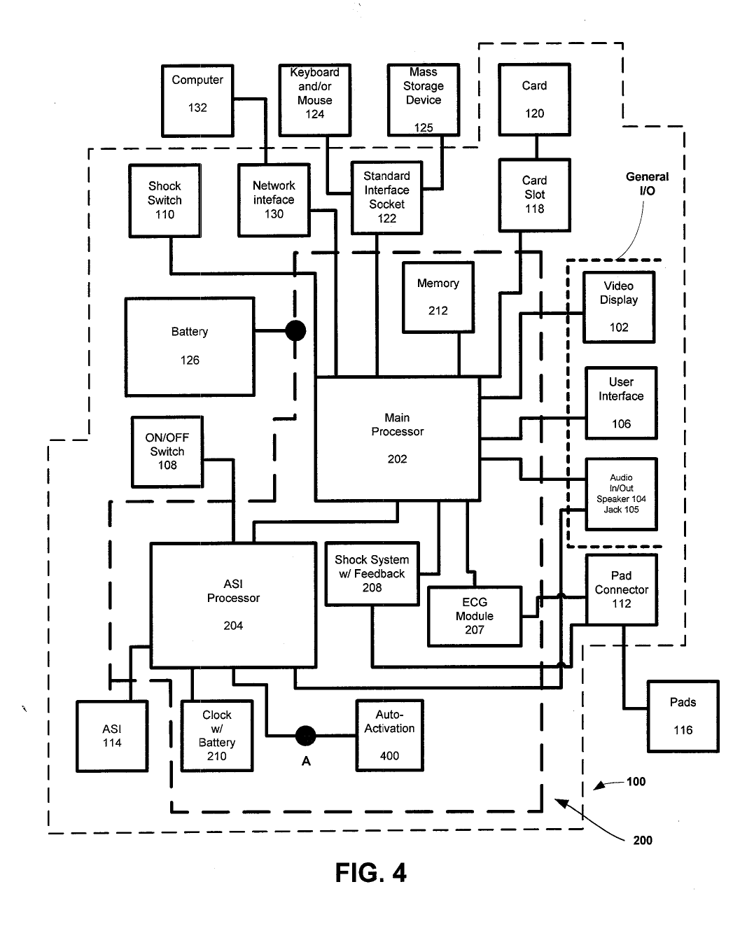

FIG. 4 is a functional block diagram of the AED 100. In addition, FIG. 4 also shows the interface of the AED 100 with various accessory components, such as the pads 116, another computer 132, a keyboard and/or mouse 124, and a mass storage device 125.

The AED's 100 circuitry (collectively referred to by reference number 200) includes a main processor 202, an active status indicator (ASI) processor 204, an ECG module 207 for receiving and conditioning ECG signals received using the pads 116, and a shock system with feedback 208, which are all powered by the battery 126.

The main processor 202 and ASI processor 204 include programmable circuitry, for running programs, including rescue programming as well as other programming such as self-testing programming, stored in memory 212. As those skilled in the art of computer circuitry design will appreciate circuit design alternatives are numerous; thus, the present invention should not be considered limited by this exemplary circuitry. Rescue programming, as well as other programming, for AED's is also well understood in the art.

The AED 100 has two primary modes—STANDBY and ON. The STANDBY mode has several sub-modes including SELF-TEST and AUXILIARY. The STANDBY-SELF-TEST sub-mode is the default mode. More specifically, the AED 100 must always be in a mode. Thus, when the AED 100 is referred to as being in the STANDBY mode, it is in one of the sub-modes. When the AED 100 is in the STANDBY SELF-TEST sub-mode, from a user's perspective the AED 100 is considered OFF.

In the STANDBY SELF-TEST sub-mode, the circuitry 200 of the AED 100 utilizes minimal power to maintain basic functions of the AED such as running a clock 210 (which is shown as having a backup battery) and automatically (i.e., without human intervention) initiating self-tests, so that scheduled self-diagnostic maintenance checks in response to the passage of time are performed.

Customarily, for a rescue attempt, the AED 100 is put into the ON mode from the STANDBY SELF-TEST sub-mode by operation of the ON/OFF switch 108 (some AED programming may permit the entering the ON mode from other modes or sub-modes). In the ON mode, unlike the STANDBY sub-modes, the AED 100 circuitry 200 is capable of delivering a therapeutic shock via the pads 116 to a patient. For example, the main processor 202 controls the shock system 208. In the ON mode, the shock system 208 is charged, armed, and then may be discharged through the pads 116, if appropriate, as a result of pressing the shock switch 110. If not appropriate, the shock system 208 is autonomously internally discharged. When the ON mode is entered, the circuitry 200, however, may be checked by a self-test.

After the rescue attempt, the AED 100 may be put back into the STANDBY SELF-TEST sub-mode by operation of the ON/OFF switch 108.

As the illustrated AED 100 is of the semi-automatic type, a therapeutic shock, if deemed appropriate, is delivered by pressing the shock switch 110. As those skilled in the AED art will appreciate, the AED could be of the automatic type. In that case, rescue programming would autonomously deliver the therapeutic shock.

The main processor 202 primarily controls the AED 100 in the ON and STANDBY sub-modes, excluding the SELF-TEST sub-mode. The ASI processor 204 primarily controls the AED 100 in the STANDBY SELF-TEST sub-mode, but does perform self-tests by awaking the main processor 202 which determines the necessary self-test and retrieves and runs the applicable computer programming. The ASI processor 204, however, provides backup to the main processor 202 in the event of a failure of the self-test programming that should have run on the main processor. In other words, both the main processor 202 and the ASI processor 204 are capable of controlling the status of the active status indicator 114.

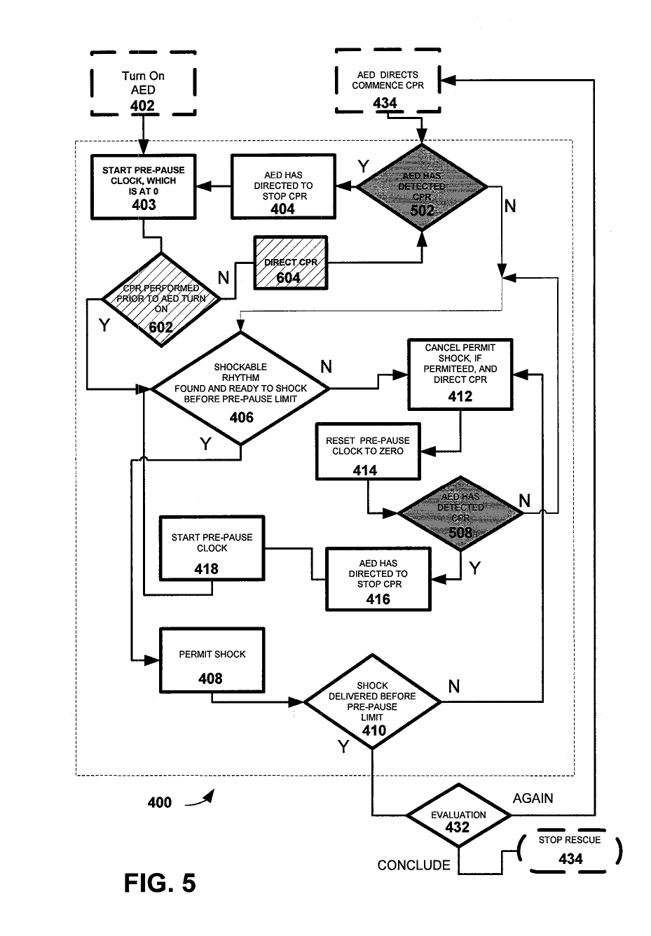

FIG. 5 shows a flowchart of a pre-shock pause period program, generally referred to by reference no. 400, is stored in the memory 212 of the AED. The pre-shock pause period program runs, at an appropriate time, concurrently with the AED's rescue programming. In this illustrative example, steps and decisions in dotted lines are considered part of the AED rescue programming. Steps and decisions having grayed out or hashed backgrounds disclose additional optional features that could be incorporated into the pre-shock pause period program.

The pre-shock pause program 400 focuses on having CPR performed and a therapeutic shock delivered within a window of time after CPR has been halted. The pre-shock pause program 400 relies on inputs from the AED's rescue programming. As those skilled in the art will appreciate, there are myriad AED rescue programs having varying features. As for this invention, the AED rescue programming at a minimum must be capable of determining whether a victim's heart rhythm is a shockable rhythm, and whether a therapeutic shock has been delivered. A highly desirable programming aspect is to be able to determine if CPR is being performed.

In this exemplary embodiment, the pre-shock pause program 400 employs a pre-pause clock, which counts intervals, e.g. seconds, to keep track of the time from when CPR is stopped. In essence, the pre-pause clock is a software stop watch that is continuously evaluated to determine if a pre-shock pause period has been exceeded.

As a general rule, the pre-shock pause program 400 contains programming that in the event the pre-shock pause period is ever exceeded, the pre-shock pause program 400 directs the AED's rescue programming to request that CPR be initiated.

That said, it is important that the pre-shock program 400 not require CPR to permit the AED rescue programming to permit a therapeutic shock. Thus, when the AED's rescue programming can make a determination as to whether CPR is being performed, in the event CPR is not started or prematurely halted, defaults in the pre-shock program should be set to assure that the pre-shock pause program permits the AED rescue programming to reach the point of delivering, or permitting, a therapeutic shock in the minimum time possible. The default sequence is a matter of design choice, therefore, the method presented should only be considered illustrative of the capability.

Continuing with an explanation of specific aspects of the pre-shock pause program 400 within the context of a rescue attempt depicted in FIG. 5, as an initial step in a rescue, the AED is turned ON, step 402.

In this basic embodiment, immediately after the AED is turned ON, the pre-pause clock will be started, step 403, and the AED rescue programming will immediately attempt to determine if the victim's heart has a shockable rhythm. The pre-shock pause program 400 will be monitoring to determine if a shockable rhythm is determined to exist within the pre-shock pause period, step 406. If a shockable rhythm is not found, the AED rescue program directs commencing CPR, step 412.

If a shockable rhythm is found within the pre-shock pause period, the pre-shock pause program 400 permits the AED rescue program to permit a therapeutic shock, step 408. The pre-pause program 400 then continues to monitor to determine if the therapeutic shock is delivered within the pre-shock pause period, step 410.

Where the AED is of an automatic type, the AED will be able to pre-determine if a therapeutic shock can be delivered within the pre-shock pause period. Thus, a NO decision is step 410 could be reached before the pre-shock pause period is actually reached. More specifically, for an automatic AED, the AED will know the time lag, at least the minimum time lag, between determining a shockable rhythm and administering a therapeutic shock. As a result, the automatic AED will be able to add the known minimum time lag to the time spent determining if the heart rhythm was shockable. If the sum of the times exceeds the pre-shock pause period, the programming does not need to wait to exit decision 410 to direct CPR.

Where the AED is of a semi-automatic type, the rescuer is directed to administer the shock using a shock button 108; thus, a rescuer could hesitate to push the shock button. As the result, the pre-shock pause period will only be reached if the shock button 108 is not pressed within the pre-shock pause period. For example, the time interval until the shock button is pushed is at least the sum of the time interval to analyze the heart rhythm and the time to press the shock button. If this exceeds say 20 seconds, CPR should be performed before the therapeutic shock is administered. Therefore, the AED programming is monitoring the pre-pause clock to determine if the therapeutic shock is delivered before expiration of the pre-shock pause period.

The pre-shock pause program 400 receives input from the AED's rescue program when the therapeutic shock has been delivered. In the event the therapeutic shock is not delivered before expiration of the pre-shock pause period, the pre-shock pause program 400 directs the AED rescue program to prompt the rescuer to perform CPR and the therapeutic shock is canceled, step 412. In accordance with current protocol, CPR should be performed for 2 minutes before the heart is reassessed as to whether a shockable rhythm is present. During the performance of CPR, the pre-pause clock is reset to zero, step 414.

At the end of the CPR period, step 416, the pre-pause clock is started, step 418, and the heart is assessed again to determine if a shockable rhythm is present, decision 406. It should be noted, that a check may be made to determine if there remains sufficient time to deliver a shock within the pre-pause period.

If a therapeutic shock is delivered, decision 410, the victim is visually accessed to determine if an additional shock is required (i.e., another treatment cycle), decision 432. If no further shocks are needed, the treatment is concluded. If the treatment is not concluded, the AED programming directs CPR, step 434. As those skilled in the AED art will appreciate, generally AEDs default to providing another treatment cycle (the number of treatment cycles, however, may be fixed), and it is a user that terminates treatment by instructing the AED to stop (e.g., turning the AED off).

As those skilled in the art will appreciate, the number of times an AED goes through the sequence may be fixed, such as three times. In addition, even if the shock treatment is deemed a success, the AED may continue monitoring the heart to determine if it relapses.

A first variation of the pre-pause program is shown in FIG. 5 by adding decisions points 502 and 508. This variation is only possible if the AED programming is capable of determining if CPR is actually being performed. For this variation, the AED programming indicates to the pre-pause program 400 if directed CPR is actually being performed. In the event CPR is not being performed, the program directs the AED program to determine immediately if a shockable rhythm is present, step 406.

A second variation of the pre-pause program is shown in FIG. 5 by adding decision 602 and step 604. This variation is attempting to determine if CPR had been performed prior to the AED being turned ON. For the illustrative AED, this could be determined by asking a question, orally (speaker 104) or visually (video display 102). The question could be answered using the user interface 106.

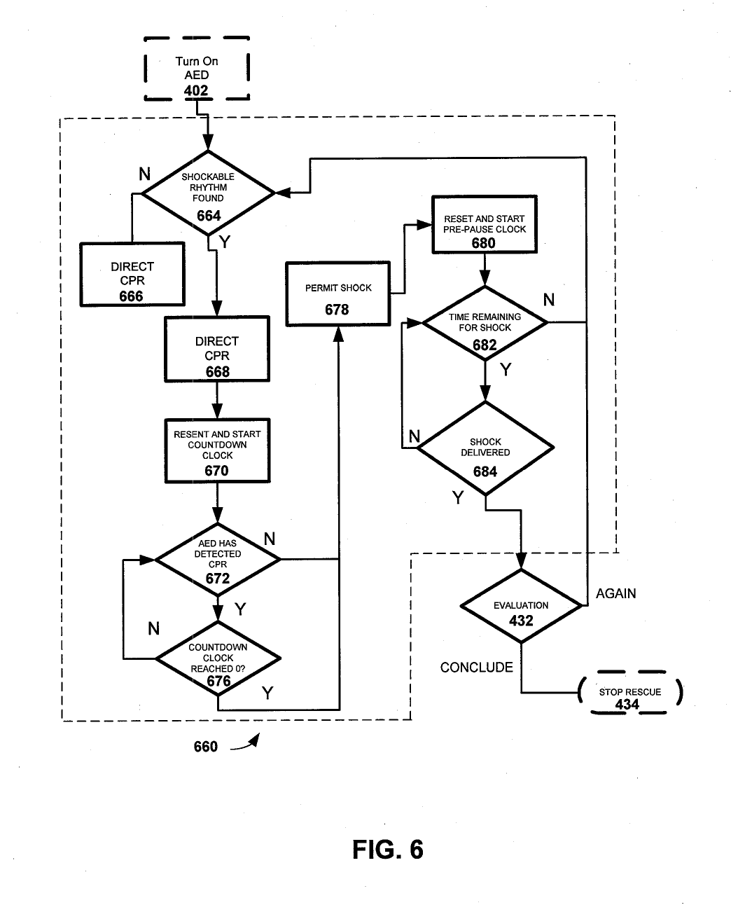

Continuing with FIG. 6, in this second embodiment of the pre-shock pause program, generally referred to by reference number 660, CPR is directed prior to a shock being permitted.

In accordance embodiment, the AED is turned ON, step 402. The program then waits to determine if a shockable rhythm has been found, step 664. In the event a shockable rhythm is not found, the program directs customary AED programming to terminate the rescue and direct CPR.

Upon finding a shockable rhythm, the programming receives input that the AED programming has directed that CPR be performed, step 668 and a countdown clock set to zero and started, step 676. Present standards indicate that a minimum therapeutic amount of CPR is around 1 minute. Therefore, the countdown clock would be set to one minute.

If the AED programming has the capability, the program determines if CPR is being performed. As long as CPR is detected, the countdown clock is monitored to determine when time has expired, step 676. If CPR is not detected, a shock is immediately authorized, step 678. In the event the AED lacks a CPR detection capability, CPR is assumed to be being performed. It should be appreciated that during this CPR period, the AED could be performing activities related to delivering a therapeutic shock, such as charging.

When time has expired, the program permits the AED to authorize a shock. The program, using a pre-pause clock, step 680, monitors to determine if a shock is delivered in a timely fashion, steps 682 and 684. As explained above, a shock is ideally delivered within a short period of time after the cessation of CPR. If a shock is not delivered in a timely fashion, the program cancels the shock and interrupts the standard CPR program instructing that it again determine if a shockable rhythm is present, step 664, and proceeds from there.

If a timely shock is delivered, standard AED programming would analyze the heart rhythm to determine if ii is normal. If the program is informed that the rhythm is normal, the rescue would be considered a success, step 434, and concluded. If not, the AED programming would start the rescue sequence over again as would the pre-pause program.

While the invention has been presented as an adjunct program receiving inputs (e.g., shock delivery, shockable rhythm detected) from conventional AED programming, this presentation has been for clarity and should not be considered limiting. More specifically, this presentation highlights the timing issues associated with the invention as those timing issues relate to conventional events contained in AED programming. As those skilled in the computer programming arts will appreciate, the programming identified above could be easily incorporated into the conventional AED programming, such that it is not an identifiable program.

While the invention has been described above by reference to various embodiments, it will be understood that many changes and modification can be made without departing from the scope of the invention. For example, it should be appreciated that a countdown clock could be used in lieu of stop watch, or visa versa, with appropriate program modifications (e.g., instead of setting to zero as for a stop watch the countdown clock would be set to the maximum permitted time, such as 20 seconds, and then reset to that time when appropriate). In addition, the exemplary AED is of the semi-automatic type, but the invention is equally relevant to an automatic type. While for an automatic type rescuer delay is not an issue, the ability to automatically deliver a shock in a timely fashion may not occur. The outputs from the AED rescue programming, if not present, can be added to the AED rescue programming as the AED rescue programming needs these inputs for other customary functions. It is therefore intended that the foregoing detailed description be understood as an illustration of the presently preferred embodiments of the invention, and not as a definition of the invention. It is only the following claims, including equivalents, which are intended to define the scope of this invention.

What is claimed is:

1. A method of delivering a therapeutic shock to a victim of cardiac arrest comprising the steps of:
establishing a pre-shock pause period;
determining if a victim's heart has a shockable rhythm;
if a shockable rhythm is found, authorizing a therapeutic shock; and
if a therapeutic shock is authorized, determining if the therapeutic shock is delivered before expiration of the pre-shock pause period, and if not, directing CPR for a period of time prior to authorizing a next therapeutic shock.

2. The method of claim 1 wherein the pre-shock pause period is less than 20 seconds.

3. The method of claim 1 wherein the pre-shock pause period is less than 10 seconds.

4. The method of claim 1 further including the step of after directing CPR, monitoring to determine if CPR is being performed.

5. The method of claim 4 further including the step of in the event CPR is not being performed, concluding performance of the step prior to the end of the period of time.

6. An AED capable of delivering a therapeutic shock to a victim, comprising:
an AED unit having circuitry with programming thereon including programming implementing the method of claim 1.

7. The AED on claim 6 further including a pair of pads electrically connected to the AED unit for conveying a therapeutic shock from the AED unit to a victim.

8. A method of delivering a therapeutic shock to a victim of cardiac arrest comprising the steps of:
analyzing a victim's heart rhythm to determine if the heart rhythm is a shockable rhythm;
after making a determination of a shockable rhythm, directing the performance of CPR for a period of time;
immediately at the end of the period of time, authorizing a shock, and administering the shock.

9. The method of claim 8 further including the step of after directing CPR, monitoring to determine if CPR is being performed.

10. The method of claim 9 further including the step of in the event CPR is not being performed after being directed, concluding performance of the step directing CPR prior to the end of the period of time.

11. An AED capable of delivering a therapeutic shock to a victim, comprising:
an AED unit having circuitry with programming thereon including programming implementing the method of claim 8.

12. The AED on claim 11 further including a pair of pads electrically connected to the AED unit for conveying a therapeutic shock from the AED unit to a victim.
